(19) United States
(12) Patent Application Publication
(10) Pub. No.: US 2007/0299046 A1
(43) Pub. Date: Dec. 27, 2007

(54) Orally available light-independent antineoplastic compounds

(76) Inventors: Mai Nguyen Brooks, Westlake Village, CA (US); Andrew John Norris, Los Angeles, CA (US)

Correspondence Address: Craig A. Crandall, APC, 3034 Deer Valley Avenue, Newbury Park, CA, 91320, US

(21) Appl. No.: 11/474,890

(22) Filed: Jun. 26, 2006

(51) Int. Cl. A61K 31/409; C07D 487/22; A61K 31/555

(52) U.S. Cl. 514/185; 540/145; 514/410

(57) ABSTRACT

Pheophorbide derivative compounds which can inhibit cell proliferation and angiogenesis in a light-independent manner are disclosed and claimed. Importantly, these compounds exhibit low toxicity, and are orally/subcutaneous/intravenously/transdermally/topically available, thus having value as new potential agents to treat cancer or diseases related to imbalance in cell proliferation and angiogenesis.

### TECHNICAL FIELD

The field of the present invention relates to the development of novel antineoplastic compounds. More specifically, the present invention provides pheophorbide derivative compounds which can prevent tumor growth in a light-independent manner, which exhibit low toxicity, and which are orally available.

### BACKGROUND OF THE INVENTION

Breast cancer is the most common cancer and is the second leading cause of cancer death in women in the United States. In 2005, approximately 212,930 patients were diagnosed with breast cancer, and an estimated 40,870 died of this disease (Jemal et al., CA: A Cancer Journal for Clinicians, vol. 55, p. 10-30, 2005). The etiology of breast cancer is somewhat understood with heredity, age, ethnicity, hormonal factors, growth factors, obesity, dietary habits and environmental exposures implicated in separate studies using epidemiological methods and experimental animal models (Nixon et al., in: Heber D et al., eds. Nutritional oncology. Academic Press: San Diego, p. 447-52, 1999). Although there have been many advances in the treatment of breast cancer, the mortality from invasive and metastatic disease has not improved significantly over the past few decades (Wood et al., in: DeVita et al., eds. Cancer: principles and practice of oncology. Lippincott-Raven: Philadelphia, p. 1415-77, 2005). Early diagnosis of breast cancer has contributed substantially to the recently reported higher survival of this disease, and has resulted in cures for selected patients. It has also become clear that the prevention of breast cancer is a crucial component of any rational effort towards the control of this deadly disease. Furthermore, in those patients who have survived breast cancer, it is important to identify measures that would keep them in long-term clinical remission.

Towards the goal of prevention, many studies have been launched in order to identify effective chemopreventive agents. Recent advances in the understanding of the mechanisms of carcinogenesis have led to the synthesis of new drugs that can inhibit tumor development in experimental animals by selective action on specific molecular targets, such as the estrogen, androgen, and retinoid receptors or inducible cyclooxygenase (Howe et al., Seminars in Oncology, vol. 29(3S11), p. 111-19, 2002). In the field of breast cancer, chemoprevention in the form of selective estrogen receptor modulators such as tamoxifen (Fisher et al., Journal of the National Cancer Institute, vol. 90, p. 1371-88, 1998) and raloxifene (Buzdar et al., Clinical Cancer Research, vol. 12, p. 1037s-48s, 2006) has generated much interest. Other effective breast cancer prevention strategies for high risk women include prophylactic mastectomy (Anderson, Breast Journal, vol. 7, p. 321-30, 2001) and/or oophorectomy. However, these expensive methods are often undesirable to patients as surgery is disfiguring and traumatic, and long term tamoxifen administration involves serious side effects. As such, there have been extensive efforts to identify alternative methods to prevent breast cancer and/or reduce the risk.

Elements in the diet have been implicated in cancer causation and the progression of established tumors. Phytochemicals in edible plants have preventive benefits through antioxidation and via gene-nutrient interactions. A number of plant chemicals or nutritional elements including resveratrol from grapes and red wine (Mollerup et al., International Journal of Cancer, vol. 92, p. 18-25, 2001), lycopene from tomatoes (Giovannucci et al., Journal of the National Cancer Institute, vol. 87, p. 1767-76, 1995) and catechins from green tea (Sartippour et al., Nutrition and Cancer, vol. 40, p. 149-56, 2001) have been considered as potential chemopreventive agents based on epidemiological observations and laboratory experimental studies

Within the area of cancer therapeutics, natural products continue to be the most abundant source of chemotherapeutic agents. Within the period spanning 1981-2002, a recent survey shows that of the 79 NCE's approved for use against cancer, 74% are either natural products, are based thereon, or mimicked natural products in one form or another (Newman et al., Journal of Natural Products, vol. 66, p. 1022-37, 2003). One example of a plant-derived product is paclitaxel (currently marketed as TAXOL® by Bristol-Myers Squibb Oncology Division). Paclitaxel is a natural diterpene product isolated from the Pacific yew tree (Taxus brevifolia). It is a member of the taxane family of terpenes. It was first isolated in 1971 by Wani et al. (Journal of the American Chemical Society, vol. 93, p. 2325-7, 1971), and one mechanism for its activity relates to its capacity to bind tubulin, thereby inhibiting cancer cell growth (Schiff et al., Nature, vol. 277, p. 665-7, 1979; Kumar, Journal of Biological Chemistry, vol. 256, p. 10435-41, 1981). Paclitaxel is effective as chemotherapy for several types of neoplasms including breast (Holmes et al., Journal of the National Cancer Institute, vol. 83, p. 1797-805, 1991).

In addition to the above approaches, it has been proposed first by Folkman that anti-angiogenic drugs may be useful in the prevention and treatment of cancer (New England Journal of Medicine, vol. 285, p. 1182-6, 1971). Much experimental evidence has demonstrated that the growth and metastasis of solid tumors in general, and breast cancer in particular, are dependent on their ability to initiate and sustain new capillary growth, i.e. angiogenesis (Folkman, Experimental Cell Research, vol. 312, p. 594-607, 2006). Blood vessel density, a marker of angiogenesis, has been shown to have prognostic significance in breast cancer (Weidner et al., New England Journal of Medicine, vol. 324, p. 1-8, 1991). Moreover, cancer cells are known to secrete potent angiogenic growth factors and the levels of these factors in the serum and urine of cancer patients has been shown to correlate with disease status (Nguyen et al., Journal of the National Cancer Institute, vol. 86, p. 356-61, 1994; Nguyen, Investigative New Drugs, vol. 15, p. 29-37, 1997; Liu et al., Lancet, vol. 356, p. 567, 2000; Sartippour et al., Cancer Epidemiology Biomarkers and Prevention, vol. 14, p. 2995-8, 2005). Therefore, interruption of angiogenesis with non-cytotoxic compounds would be a treatment approach ideally suited for many breast cancer patients, as well as for patients at high risk of developing breast cancer.

Multiple agents have been developed in order to inhibit the phenomenon of tumor-induced angiogenesis. Many of these drugs are in human clinical trials (deCastro et al., ePub, Apr. 3, 2006). The drug Avastin®, an inhibitor of the potent angiogenic factor VEGF (vascular endothelial growth factor) has recently been approved by the USFDA (United States Food and Drug Administration) for the treatment of colorectal cancer (Kabbinavar et al., Journal of Clinical Oncology, vol. 21, p. 60-5, 2003). Other drugs tested include TNP-470, CM-101, interferon-α, IL-12, platelet factor-4, pentosan polysulfate, tecogalan, suramin, antibodies against VEGF or integrins, and VEGF receptor antagonists (Lee et al., in Mousa S A, ed. Therapeutic implications of angiogenesis inhibitors and stimulators: Current status and future treatment directions. Landes Bioscience: Texas, p. 151-60, 2002).

Unfortunately, a major shortcoming of the vast majority of the anti-angiogenic drugs under development, as well as chemotherapeutic drugs such paclitaxel, is the fact that they cannot be effectively administered by the oral route to human patients because of poor or inconsistent systemic absorption from the gastrointestinal tract. For example, paclitaxel is very poorly absorbed when administered orally (less than 1%); see, e.g., Eiseman et al., Second NCI Workshop on Taxol and Taxus (September 1992) and Suffness (ed., Taxol Science and Applications, CRC Press: Boca Raton, 1995). Eiseman et al. indicated that paclitaxel has a bioavailability of 0% upon oral administration, and Suffness et al. reported that oral dosing with paclitaxel did not seem possible since no evidence of antitumor activity was found on oral administration up to 160 mg/kg/day. For this reason, paclitaxel has not been administered orally to human patients in the course of treating paclitaxel-responsive diseases. These drugs are, therefore, generally administered via intravenous or subcutaneous routes, potentially requiring intervention by a physician or other health care professional, entailing considerable discomfort and potential local trauma to the patient and even requiring administration in a hospital setting with surgical access in the case of certain IV infusions. This is particularly problematic for the anti-angiogenic drugs, since they need to be given on a long-term basis in order to control cancer growth, due to their cytostatic rather than cytotoxic properties. Moreover, many of the anti-angiogenic drugs and other pharmacologically active compounds, including the recently reported potent angiogenic inhibitors angiostatin and endostatin, are complex molecules that are difficult and expensive to produce in the quantities and purities required for human use. Clearly, there still exists a need for less complex, less expensive, orally available cancer therapeutics.

With a goal of identifying a potent anti-angiogenic drug that could be administered orally, the present inventors initiated a screening program designed to test a wide variety of plant extracts of Asian descent for anti-angiogenic and anti-tumor activity. The choice of these plant extracts was based upon anecdotal success in traditional Oriental medicine that has been practiced for centuries in Asian countries (Huang, in: Huang, ed. The pharmacology of Chinese herbs. CRC Press: Boca Raton, p. 457-83, 1999). One such plant is Livistona chinensis, and the extract from the seed of Livistona chinensis has potent anti-tumor activity and is present as an ingredient in many Oriental anti-cancer regimens (Liu et al., Bulletin of the Institute of Zoology Academia Sinica, vol. 26, p. 143-50, 1987). In a previous report by the present inventors, it was hypothesized that the Livistona chinensis extract inhibits tumorigenesis and angiogenesis, and that the identification of active component(s) in Livistona chinensis is critical to the chemopreventive or chemotherapeutic application of this extract (Sartippour et al., Oncology Reports, vol. 8, p. 1355-7, 2001). To date, the literature has yet to reveal studies that have been carried out to analyze in depth the active components of the Livistona chinensis seed and the active component(s) of Livistona chinensis is/are unknown.

As described in further detail herein, the present inventors have isolated and identified the active component(s) from the extract of the Livistona chinensis seed. Using a variety of chemical and analytical techniques, including solvent extraction, chromatographic separation, nuclear magnetic resonance, photo diode array, infrared spectroscopy and high-resolution mass spectrometry, the complete chemical structure was identified and it was determined that the active compound was a pheophorbide-α derivative.

As related to pheophorbide-α derivatives, it has been reported previously that certain pheophorbide-α derivatives, such as pheophytin-α and pheophorbide-α methyl esters, inhibit replication of a hepatoma tissue culture (HTC) cell line following light irradiation (Nakatini et al., Chemical and Pharmaceutical Bulletin, vol. 29, p. 2261-9, 1981). These pheophorbide-α derivatives are structurally related to porphyrin compounds, and porphyrin compound derivatives are known for their binding property with cancer tissue and photo-dynamic characteristics and are widely used in photodynamic therapy (PDT). PDT is a modality developed to treat cancer with a combination of light and photosensitizers (Henderson et al., eds. Photodynamic therapy, basic principles and clinical applications. Marcel Dekker: New York, 1992). U.S. Pat. No. 4,709,022 by Sakata et al. (the '022 patent) discloses pheophorbide derivatives and alkaline salts thereof useful as novel photosensitizers in cancer treatment, including PDT. U.S. patent application Ser. No. 11/059,557 by Robinson (the '557 application) discloses substituted porphyrin derivatives suitable as pharmaceutical agents for use in PDT, MRI diagnosis, and radiodiagnostics. Other photosensitizers are disclosed in U.S. Pat. Nos. 5,633,275 by Mori et al.; 5,654,423 by Kahl et al.; 5,675,001 by Hoffman et al.; 5,703,230 by Boyle et al.; and 5,705,622 by McCapra). One such photosensitizer, Photofrin (U.S. Pat. No. 4,882,234 by Lai et al.) is approved by the USFDA to treat esophageal and endobronchial non-small cell lung cancers. It has been used anecdotally as PDT of breast cancer chest wall recurrence after mastectomy (Cuenca et al., Annals of Surgical Oncology, vol. 11, p. 322-7, 2004). A second generation sensitizer Photochlor was designed as an alternative, to lessen the prolonged and sometimes severe cutaneous phototoxicity associated with Photofrin, and used in a clinical trial (Bellnier et al., Cancer Chemotherapy and Pharmacology, vol. 57, p. 40-5, 2006).

A major problem in the pharmaceutical application of porphyrins and/or pheophorbide-α derivatives is their low solubility in physiological solutions, rendering it nearly impossible to prepare effective pharmaceutical grade injectable solutions (see, e.g., U.S. Pat. Nos. 5,378,835 by Nakazato (the '835 patent) and 6,777,402 by Nifantiev et al. (the '402 patent)). To address this problem, the '835 patent discloses a method for producing a water-soluble pheophorbide-α that can be safely used in humans, and the '402 patent discloses high purity pharmaceutical-grade water-soluble porphyrin derivatives useful as photosensitizers for PDT of cancer, infectious and other diseases as well as for light irradiation treatments in other areas. There is no discussion in any of patents referenced above relating to photo-independent pheophorbide-α derivatives or pheophorbide-α derivatives that are orally available.

There have been published reports disclosing pheophorbide-related compounds which appear to be photo-independent. For example, Cheng et al. (Journal of Natural Products, vol. 64, p. 915-9, 2001) disclosed cytotoxic pheophorbide-related compounds isolated from the leaves and stems from Clerodendrum calamitosum and C. cyrtophyllum and identified several extracts as potent cytotoxic agents against seven tumor cell lines without direct illumination. Cheng et al. hypothesized that the cytotoxic effect of the compounds may occur through mechanisms other than photodynamic action such as metal dependent DNA cleavage. Cheng et al. did not describe the pheophorbide derivative compounds of the present invention as being photo-independent. Likewise, Wongsinkongman et al. (Bioorganic and Medicinal Chemistry, vol. 10, p. 583-91, 2002) disclosed pheophorbide-α derivatives as photo-independent cytotoxic agents. Importantly, Wongsinkongman et al. disclosed that only certain metal analogues of pheophorbide-α were found to exhibit potent but essentially photo-independent activity in vitro. Neither Cheng et al. or Wongsinkongman et al. disclosed any orally available pheophorbide-α derivative compounds nor did they demonstrate any light independent in-vivo activity. Nakamura et al. (Cancer Letters, vol. 108, p. 247-55, 1996) disclosed the inhibitory effect of pheophorbide-α derivatives on skin tumor promotion in an ICR mouse skin model. Nakamura et al. disclose that pheophorbide-α derivatives reduce tumor promotion initiated by inflammatory compounds which are phorbol esters (e.g. TPA). Nakamura et al. proposed that the pheophorbide-α derivatives act via an anti-inflammatory mechanism of action rather than an antioxidant mechanism of action.

At the current time, there are only a handful of approved oral drugs for treatment of any and all solid tumors and hematologic malignancies. The discovery of novel anti-tumor agents that can be given by mouth, the most simple and least morbid route of administration, is of tremendous importance in a disease that is known as the number one killer in America. Such a discovery would have tremendous impact in the efforts to decrease the pain and suffering caused by cancer and its associated treatments. The present inventors describe the preparation of pheophorbide derivative compounds, and formulations thereof, which have low toxicity, can prevent tumor growth in a light-independent manner, and which are orally available. As such, these pheophorbide derivative compounds have great importance as potential antineoplastic agents that will be inexpensive and which can be administered orally to large populations as potential therapy for cancer, or possibly as a dietary supplement for chemoprevention, over a period of several years in order to prevent cancer initiation and/or to keep existing microscopic tumors dormant; thus providing tremendous benefit to individuals having to deal with cancer and other cell proliferation disorders.

### SUMMARY OF THE INVENTION

One aspect of the present invention is to provide pheophorbide derivative compounds having the general Formula I,

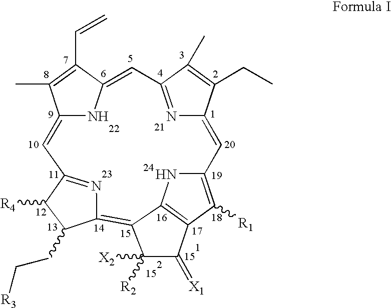

wherein R₁═R₄═CH₃; R₂═CH₂OH, COOH, CHO, COOCH₃, alkyl or ester or ether linked alkyl group containing alkyl substitutents such as (CH₂)ₙCH₃ where n is between 0 and 24 and may contain 0, 1, 2 or 3 double bonds and 0 or more hydroxy moieties, or may contain, but is not limited to, one or more of the following esters such as hemisuccinate, choline, phosphate, phosphoryloxymethylcarbonyls, amino acid, dimethylaminoacetate, phosphonate, and N-alkoxycarbonyl or phosphoryloxymethyloxycarbonyl derivatives; R₃═OH, COOH, COOCH₃, alkyl group or ester linked alkyl group containing alkyl substitutents such as (CH₂)ₙCH₃ where n is between 0 and 24 and may or may not contain 0, 1, 2 or 3 double bonds and 0 or multiple hydroxy moieties, or may contain, but is not limited to, one or more of the following esters such as hemisuccinate, choline, phosphate, phosphoryloxymethylcarbonyls, amino acid, dimethylaminoacetate, phosphonate, and N-alkoxycarbonyl or phosphoryloxymethyloxycarbonyl derivatives; X₁═O, S, or the reduced form at C15¹ yielding OH, SH, and any ester or ether derivatives thereof; X₂═H, OH, OCH₃, OCH₂CH₃, OAc, SH, Cl, F; and wherein said compounds are photo-independent cytotoxic agents able to inhibit tumor growth in a mammal.

Another aspect of the present invention relates to a pharmaceutical composition, and method of preparing said pharmaceutical composition, wherein said composition comprises at least one pheophorbide derivative compound as an active ingredient, in a pharmaceutically acceptable excipient, carrier or vehicle. One preferred method of preparing said composition generally comprises the steps of: obtaining a pure powder composition comprising the active ingredient; adding a detergent or carrier agent in a neat solvent of which said powder is readily dissolved to form a mixture; drying said mixture to form a dried mixture; and resuspending said dried mixture in a biologically compatible solvent to form a fully reconstituted composition.

Another aspect of the present invention relates to a pharmaceutical composition for oral administration to a mammalian subject, comprising: a) at least one pheophorbide derivative compound as active ingredient; and b) a vehicle comprising a detergent containing a minimum of 1% and a maximum of 30% by volume of a carrier comprising TWEEN-80 or a like detergent with both components dissolved in distilled sterile water, or a biologically compatible solvent such as PBS or normal saline.

Another aspect of the present invention relates to chemically modified derivatives of Formula I that are intended to make the compound more soluble in water for their application in in vivo use for light independent anti tumor therapy.

Another aspect of the present invention relates to providing an efficient and convenient method for obtaining a compound having therapeutic activity from a plant or plant part. The method comprises the steps of: obtaining the raw material, e.g., seeds, from the plant; extracting or exuding compounds from the raw material; identifying active compounds using in vitro assays; separating active compounds using flash column chromatography; identifying active compounds using in vivo assays; fractionating active compounds using HPLC; screening active compounds using in vitro assays; purifying active compounds using high resolution chromatography; and identifying active compounds via a final in vivo assay.

Another aspect of the present invention relates to a method of treating tumors or tumor metastases in a patient, comprising: administering to said patient a therapeutically effective amount of a pharmaceutical composition comprising at least one pheophorbide derivative compound in pharmaceutically acceptable excipient, carrier or vehicle.

### BRIEF DESCRIPTION OF THE DRAWINGS

FIGS. 1A and 1B depict the effect of Livistona inner kernel vs. whole seed extract on cancer cell proliferation in human breast cancer cell line MDA-MB-231 (FIG. 1A) and human breast cancer cell line MCF-7 (FIG. 1B). Extracts are expressed as percentage (%) of media volumes. Data represents the mean±SE (standard error) of three different values: * P<0.05, ** P<0.01 and *** P<0.001 compared to positive control (CTL)(media with serum). CTL⁻ is a negative control (media without serum). The FIGS. 1A and 1B data show that the major inhibitory effects of Livistona derives from the shell, and not from the kernel portions.

FIG. 2A depicts the effect of Livistona inner kernel vs. whole seed extract on endothelial cell proliferation in a HUVEC (human umbilical vein endothelial cell) proliferation assay. FIG. 2B depicts the effect of Livistona complete shell vs. the shell inner thin membrane alone on endothelial cell proliferation in a HUVEC (human umbilical vein endothelial cell) proliferation assay. Extracts are expressed as percentage (%) of media volumes (FIG. 2A), and as weight per volume (FIG. 2B). Data represents the mean±SE of three different values: * P<0.05, ** P<0.01 and *** P<0.001 compared to positive control (CTL)(media with serum and growth factors). CTL⁻ is negative control (media and serum without growth factors). The FIGS. 2A and 2B data show that the highest suppressive activity resides in the inner membrane of the Livistona shell.

FIG. 3 depicts the effect of Livistona extract on human breast cancer xenograft tumors. SCID mice were injected with 10⁷ MDA-MB-231 cells subcutaneously in the flank. Control mice (n=4) then drank water and experimental mice (n=4) drank Livistona crude extract diluted with drinking water at a 1:4 ratio. Tumor volume in control mice (CTL) and experimental mice (Livistona) was measured on various days after injection. Data represents the mean±SE, * P<0.05, compared to control. The FIG. 3 data shows smaller human breast cancer xenograft tumors in the mice that drank Livistona crude extract.

FIG. 4 depicts the effect of three purified fractions of Livistona extracts on human breast cancer xenograft tumors. SCID mice were injected with 107 MDA-MB-231 cells subcutaneously in the flank. Groups of four (4) mice were then administered (by gavage) oral preparations of 40 mg/kg of Fraction A, 10 mg/kg of Fraction B, 40 mg/kg of the acetonitrile extract, and vehicle alone (CTL)(distilled water with 5% TWEEN-80). Tumor volume was measured on various days after injection. Data represents the mean±SE, * P<0.05, compared to control. The FIG. 4 data shows the smallest human breast cancer xenograft tumors in the mice administered Fraction B.

FIG. 5 depicts the effect of a particular purified Livistona extract fraction on human breast cancer xenograft tumors. SCID mice were injected with 10⁷ MDA-MB-231 cells subcutaneously in the flank. Two routes of entry were then used to deliver the extract. Oral administration was by gavage at 10 mg/kg five days per week for four weeks, and subcutaneous injection was administered at 3 mg/kg three days per week for four weeks. Tumor volume was measured on day 29. Data represents the mean±SE, * P<0.05, compared to control (CTL)(oral distilled water and 5% TWEEN-80). The FIG. 5 data shows smaller human breast cancer xenograft tumors in the mice receiving oral or subcutaneous administration of the purified Livistona extract fraction.

FIG. 6 depicts the ¹H Proton spectra of a pheophorbide derivative compound (referred to herein as AM-101) in neat deuterated DMSO having the principle Formula I wherein R₁═R₄═CH₃, R₂═CH₂COOCH₃, R₃═COOH, X₁═O and X₂═OH.

FIG. 7 depicts the effect of AM-101 tested on the human breast cancer cell line MDA-MB-231 for its inhibitory activity. Various concentrations (in μM) of AM-101 were tested in the CellTiter 96-Aqueous non-radioactive cell proliferation assay from Promega (Madison, Wis.). Data represents the mean±SE of five different values, after background was subtracted, * P<0.05, compared to wild type (WT)(cells in growth media) or to control (CON)(cells in growth media plus 0.02% TWEEN-80). The AM-101 drugs were delivered in growth media plus 0.02% TWEEN-80. The FIG. 7 data shows that the AM-101 compound of the present invention powerfully inhibits the proliferation of human breast cancer cells in the absence of light.

### DETAILED DESCRIPTION OF THE INVENTION

As those in the art will appreciate, the foregoing detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

As used herein, the term “cancer” in a mammal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

As used herein, the term “therapeutically effective amount” or “effective amount” means an amount sufficient to effect beneficial or desired results. An effective amount can be administered in one or more administrations.

As used herein, the terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the terms “anticancer agent,” “conventional anticancer agent,” or “cancer therapeutic drug” refer to any therapeutic agents (e.g., chemotherapeutic compounds and/or molecular therapeutic compounds), radiation therapies, or surgical interventions, used in the treatment of cancer (e.g., in mammals).

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs).

As used herein, the term “derivative” of a compound refers to a chemically modified compound wherein the chemical modification takes place either at a functional group of the compound, aromatic ring, or carbon backbone; including, for example, esters of alcohol-containing compounds, esters of carboxyl-containing compounds, amides of amine-containing compounds, amides of carboxyl-containing compounds, imines of amino-containing compounds, and the like.

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases well known to those skilled in the art.

As used herein, the term “administration” refers to the act of giving a drug, prodrug, or other agent, or therapeutic treatment (e.g., radiation therapy) to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, into cerebrospinal fluid, etc.) and the like.

As used herein, the term “pharmaceutically acceptable excipient, carrier, or vehicle” is intended to include any and all solvents, dispersion media and the like which may be appropriate for the desired route of administration of the pharmaceutical preparation and which are suitable for use in a human or other mammal. Except insofar as any conventional excipient, carrier or vehicle is incompatible with the pheophorbide derivative compounds described herein, its use in the pharmaceutical preparations of the invention is contemplated.

The present invention provides pheophorbide derivative compounds having the general Formula I,

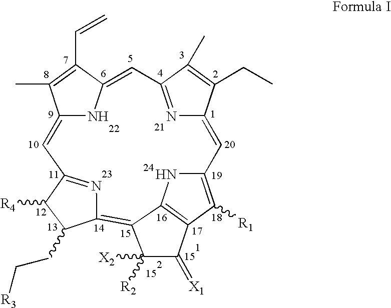

wherein R₁═CH₃; R₂═CH₂OH, COOH, CHO, COOCH₃, alkyl or ester or ether linked alkyl group containing alkyl substitutents such as (CH₂)ₙCH₃ where n is between 0 and 24 and may contain 0, 1, 2 or 3 double bonds and 0 or more hydroxy moieties, or may contain, but is not limited to, one or more of the following esters such as hemisuccinate, choline, phosphate, phosphoryloxymethylcarbonyls, amino acid, dimethylaminoacetate, phosphonate, and N-alkoxycarbonyl or phosphoryloxymethyloxycarbonyl derivatives; R₃═OH, COOH, COOCH₃, alkyl group or ester linked alkyl group containing alkyl substitutents such as (CH₂)ₙCH₃ where n is between 0 and 24 and may or may not contain 0, 1, 2 or 3 double bonds and 0 or multiple hydroxy moieties, or may contain, but is not limited to, one or more of the following esters such as hemisuccinate, choline, phosphate, phosphoryloxymethylcarbonyls, amino acid, dimethylaminoacetate, phosphonate, and N-alkoxycarbonyl or phosphoryloxymethyloxycarbonyl derivatives; X₁═O, S, or the reduced form at C15¹ yielding OH, SH, and any ester or ether derivatives thereof; X₂═H, OH, OCH₃, OCH₂CH₃, OAc, SH, Cl, F; and wherein said compounds are photo-independent cytotoxic agents able to inhibit tumor growth in animal or humans. In one preferred pheophorbide derivative compound of the present invention, R₁═R₄═CH₃, R₂═CH₂COOCH₃, R₃═COOH, X₁═O and X₂═OH.

In some embodiments of the present invention, the pheophorbide derivative compounds are derived from plants. The plants contemplated for use in the present invention may be any one of a wide variety of plants and may be sexually or vegetatively propagated plants as is further described herein. In particular, plants suitable for use in the invention, such as use in the method for eliciting a compound having therapeutic activity as described below, include: Livistona chinensis, Neptunia oleracea, Clerodendrum calamitosum, Clerodendrum cyrtophyllum, Atropa bella donna, Erythrina flabelliformis, Ipomoea tricolor, Erythrina crista, Celosia cristata, Gallium spurium, Laurus nobilis, Vitis labrusca, Vitis vinifera, Gratiola officinalis, Symphitum officinalis, Hosta fortunei, Cassia hebecaipa, Thalictrum flavum, Scutellaria altissima, Portulacca oleracea, Scutellaria certicola, Physalis sp., Geum fauriei, Gentiana tibetica, Linum hirsutum, Aconitum napellus, Podophyllum emodii, Thymus cretaceus, Carlina acaulis, Chamaecrista fasciculata, Pinus pinea, Peganumharmala, Tamarindus indica, Carica papaya, Cistus incanus, Capparis spinosa, Cupressus lusitanica, Diospyros kaki, Eryngium campestre, Aesculus woerlitzensis, Aesculus hippocastanum, Cupressus sempervirens, Celtis occidentalis, Polygonum cuspidatum, Elaeagnus angustifolia, Elaeagnus commutata, Gentiana macrophylla, Brassica rapa, Sesbania exaltata, Sesbania speciosa, Spartinapotentifiora, Brassica juncea, Helianthus annuus, Poinsettiasp., Pelargonium zonale, Synapsis sp., Leontopodium alpinum, Lupinus luteus, Buxus microphylla var. japonica, Liatris spicata, Primulajaponica, Betula nigra, Filipendula vulgrais, Lobelia siphilitica, Grevillea robusta, Reseda luteola, Gentiana littoralia, Campanula carpatica, Ageratum conizoides, Psidium guajava, Ailanthus altissima, Hydrocotyle asiatica, Brugmansia suaveolens, Thymus pulegioides, Thymus lema-barona, Thymus serphyllum (wild), Gaultheria procumbens, Thymus camosus, Thymus thracicus, Calycanthus floridus, Zin giber officinalis, Lamium dulcis, Thymus praecox “arcticus”, Thymus speciosa, Thymus pseudolamginosus, Thymus vulgraris, Ficus religiosa, Forsythia suspensa, Chelidonium majus, Thymus wooly, Thymus portugalense, Nicotiana tabacum, Thymuscytriodorus “aureus”, Cactus officinailis, Lablab purpurea, Juglans regia, Actinidia chinensis, Hemerocallis sp., Betula pendula, Gardenia jasminoides, Taxodiumdistichum, Magnolia loebherii, Crataegus praegophyrum, Larix decidua, Thuja orientalis, Thuja ociden talis, Cupressocyparis leylandii, Pseudotsuga menziesii, Abiesfinna, Parthenocissus quinquefolia, Allium cemuum, Juniperus “blue pacific”, Taraxacum officinalis, Yucca sp., Tsuga canadensis, Ilex aquifolium, Ilex comuta, Taxus hiksii, Taxus media, Metasequoia glyptostroboides, Pinus bungi ana, Buxus sempervirens, Stewartia koreana, Prunus sp., Betula dahurica, Plantago minor Acer palmatum, Acer campestre, Cotinus coggygria, Quercus robur, Acer truncatum, Achyranthes bidentata, Allium japonicum, Carum cap sicum, Agastache mexicana, Prunella vulgaris, Tagetes minuta, Nepeta cataria, Ratibidacolumnaris, Aster novae angliae, Myrica cerifera, Pittosporum tobira, Plantago major, Pinus sylvestris, Acorus canadensis, Pierisjaponica, Pinus strobus, Trifolium pratense, Prunus serotina, Datura stramonium, Geranium maculatum, Hydrocotyle asiatica, Astragalus sinicus, Centaurea maculata, Ruschia indurata, Myrthus communis, Platanus occidentalis, Licium barba turn, Lavandula officinalis, Grevillea robusta, Hypophae rhamnoides, Filipendula ulmaria, Betula pendula, Polygonum odoratum, Brugmansia graveolens, Rhus toxi codenta, Armoracia rusticana, Ficus benjaminii, Sufflera sp., Baikiaea recurvata, Asimina triloba, Lippia dulcis, Epilobium augustifolium, Brugmansia suaveolens, Xanthosoma sagittifolium, Monstera deliciosa, Aglaonema commutatus, Dieffenbachia leopoldii, Anthurium andreanum, Syngonium podophyllum, Dracaena fragrans, Ananas comosus, Strelitzia reginae, Dieffenbachia segiune, Syngonium auritum, Dracaena sp., Haemanthuskatharinae, Anthurium altersianum, Spathiphyllum grandifiorum, Spathiphyllum cochle arispatum, Monstera pertusa, Anthurium magnificum, Anthurium hookeri, Anthurium elegans, Calathea zebrina, Yucca elephantipes, Bromelia balansae, Musa textilis, Myrthus communis, Olea oleaster, Olea europaea, Nerium oleander, Cocculus laurifolius, Microsorium punctatum, Sanseviera sp., Adansonia digitata, Boehmeria biloba, Piper nigrum, Phymatosorus scolopendria, Tumera ulmifolia, Nicodemia diversifolia, Tapeinochilos spectabilis, Rauwolfia tetraphylla, Ficus elastica, Cycas circinalis, Caryota urens, Cynnamomum zeylonicum, Aechmealuddemanniana, Phoenix zeylonica, Ficus benjamina, Ficuspumila, Murraya exotica, Trevesia sundaica, Clerodendrumspeciosissimum, Actinidia kolomikta, Paeonia lactifiora, Paeonia suffruticosa, Quercus imbricaria, Iris pallida, Portulacca olleracea, Polygonum aviculare, Iris pseudocarpus, Ailium nutans, Ailium fistulosum, Anthericum ramosum, Veratrum nigrum, Polygonumlapathifolium, Hosta lancifolia, Hosta sieboldii, Echinops sphaerocephalus, Paeonia dahurica, Inula helenium, Crambe pontica, Digitalis lutea, Baptisia australis, Aristolochia australis, Hyssopusseravschanicus, Teucrium chamaedrys, Sedum album, Heracleum pubes cens, Origanum vulgare, Cachrys alpina, Laser trilobum, Matteuccia struthiopteris, Sedum telephium, Bocconia cordata, Ajuga reptans, Thalictrum minus, Anemone japonica, Clematis rectae, Alchemilla officinalis, Potentilla alba, Poterium sangiusorba, Menispermum dauricum, Oxybaphusnyctagineus, Armoracia rusticana, Crambe cordifolia, Agrimoniaeupatoria, Anchusa officinalis, Polemoniumcaeruleum, Valeriana officinalis, Pulmonaria molissima, Stachys lanata, Coronilla varia, Platycarya grandiflora, Lavandula officinalis, Vincetoxicum officinale, Acalypha hispida, Gnetum gnemon, Psychotria nigropunctata, Psychotria metbac teriodomasica, Codiaeum variegatum, Phyllanthus grandifolius, Pterigota alata, Pachyra affinis, Sterculia data, Philodendron speciosum, Pithecellobium unguis-cati, Sanchezia nobilis, Oreopanax capitatus, Ficus triangularis, Kigeliapinnata, Pipercubeba, Laurus nobilis, Erythrina cajfra, Metrosideros excelsa, Osmanthus fragrans, Cupres sussempervirens, Jacobinia sp., Senecio platyphylloides, Tetraclinis articulata, Eucalyptus rudis, Podocarpus spinulosus, Eriobotryajaponica, Gingko biloba, Rhododendronsp., Thuja occidentalis, Fagopyrum sufruticosum, Geum macrophyllum, Magnolia kobus, Vinca minor Convallaria majalis, Corylus avellana, Berberis sp., Rosa multifiora, Ostrya carpinifolia, Ostrya connogea, Quercus rubra, Liriodendron tulipifera, Sorbus aucuparia, Betula nigra, Castanea sativa, Bergenia crassifolia, Artemisia dracunculus, Ruta graveolens, Quercus nigra, Schisandra chinensis, Betula alba, Sambucus nigra, Gentiana cruciata, Encephalartos horridus, Phlebodium aureum, Microlepia platyphylla, Ceratozamia mexicana, Stenochlaena tenuifolia, Adiantum trapeziforme, Adiantum raddianum, Lygodium japonicum, Pessopteris crassifolia, Asplenium australasicum, Agathis robusta, Osmunda regaus, Osmundastrum claytonianum, Phyllitis scolopendrium, Polystichum braunii, Cyrtomium fortune, Dryopteris flux mas, Equisetum variegatum, Athyrium nipponicum, Athyrium filix-femina, Parthenocissus tricuspidata, Ligusticum vulgare, Chamaecy parispisifera, Rosa canina, Cotinus coggygria, Celtis occidentalis, Picea schrenkiana, Cyclonia oblonga, Ulmus pumila, Euonymus verrucosus, Deutzia scabra, Mespilus germanica, Quercus castaneifolia, Euonymus europea, Securinega sufruticosa, Koelreuteria paniculata, Syring a josikaea, Zelkova carpinifolia, Abies cephalonica, Taxus baccata, Taxus cuspidata, Salix babylonica, Thuja occidentalis, Actinidia colomicta, Mahonia aquifo hum, Aralia mandschurica, Juglans nigra, Euonymus data, Prinsepia sinensis, Forsythia europaea, Sorbocotoneaster pozdnjakovii, Morus alba, Crataegus macrophyllum, Eucommiaulmifolia, Sorbus commixta, Philodendron amu rense, Comus mas, Kerria japonica, Panotia persia, Jasminum fruticans, Swidasan guinea, Pentaphylloides fruticosa, Sibiraea altaiensis, Cerasus japonica, Kolkwitzia amabilis, Amigdalus nana, Acer mandschurica, Salix tama risifolia, Amelanchier spicata, Cerasus mahaleb, Prunus cerasifera, Corylus avellana, Acer tataricum, Viburnum opulus, Syring a vulgaris, Fraxinus exelsior, Quercus trojana, Chaenomeles superba, Pinus salinifolia, Berberis vulgaris, Cotoneaster horisontalis, Cotoneaster fangianus, Fagus sylvatica, Pinuspumila, Pinus sylvestris, Berberis thunbergii, Ajuga forrestii, Anisodus acutangulus, Chinchona ledgerina, Valeriana officinalis, Peganumharmala, Chrysanthemum cineraliaefolium, Tagetes patula, Scopoliajaponica, Rauwolfia serpentine, Papaver somniferum, Capsicumfrutescens, Fumaria capreolata L., Datura stramonium, Tinospora rumphii, Tripterygium wilfordii, Coptis japonica, Salvia officinalis, Colleus blumei, Catharanthus roseus, Morinda citrofolia, Lithospermumerythrorhizon, Dioscorea deltoidea, Mueune pruriens, Mirabilis Jalapa, Boerhavia diffusa, Camptotheca acuminate, Nothapodytes foetid, Morus nigra, Symphoricarpus albus and Ophiorrhiza pumila and other chlorophyll bearing plants.

It is well understood that plant sources other than the aforementioned plants can be used as a source of pheophorbide derivative compounds and starting material that can be used to synthesize pheophorbide derivative compounds can be obtained from both natural (Van Breemen et al., Journal of Agricultural and Food Chemistry, vol. 39, p. 1452-6, 1991) and commercial sources. For example, the synthesis outlined in Smith et al. (Journal of Chemical Research, Synopses, vol. 3, p. 64-5, 1987) or Ma et al. (Tetrahedron: Asymmetry, vol. 6, p. 313-6, 1995) are feasible.

The present invention further provides pharmaceutical compositions, and methods of preparing said pharmaceutical compositions, said compositions comprising at least one pheophorbide derivative compound as an active ingredient, in a pharmaceutically acceptable excipient, carrier or vehicle. One preferred method of preparing said composition generally comprises the steps of: obtaining a pure powder composition comprising the active compound of Formula I from a plant; adding a detergent or carrier agent in a neat solvent of which said active compound is readily dissolved to form a mixture; drying said mixture to form a dried mixture; and resuspending said dried mixture in a biologically compatible solvent using sonication and rigorous mixing to form a fully reconstituted composition. Alternatively, the pure powder composition comprising the active compound can be obtained from synthetic, or semi-synthetic methods. Such methods are well known and understood in the art.

In a particularly preferred embodiment for preparing said pharmaceutical composition, the obtained pure powder composition comprising the active compound is dissolved in CHCl₃ to make a concentration spanning 1-10 mg/ml in a sterile glass tube. To this, a detergent (TWEEN-80) is added in sufficient amounts to make a 1-10% (v/v) TWEEN-80 concentration in the final sample. The solution is a homogenous and clear green in color. If multiple samples are to be prepared, the solution can be allocated to multiple tubes at this time. The mixture is then dried under Nitrogen gas to dryness. To this dried mixture is added the appropriate amount of water, buffer, or saline solution (sterile) to ¼ to ½ the final volume to be used in treatment. The preparation is then immediately sonicated under warm (60° C. or less) or cold conditions for 1-30 min as needed. Once a clear homogenous solution is reached, the appropriate amount of sterile water, buffer, or saline solution is added to make the final volume required with the TWEEN-80 concentration within, but not restricted to 1-10% (v/v) as needed. The preparation is then sonicated another 1-10 min and placed in storage till used. The final preparation is homogenous and clear in consistency.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal), intranasal, epidermal and transdermal, parenteral, or oral.

In preparing the compositions for oral dosage forms, any convenient pharmaceutical media may be employed. For example, water, glycols, oils, alcohols, flavoring agents, preservatives, coloring agents, and the like may be used to form oral liquid preparations such as suspensions, elixirs and solutions. Aqueous suspensions may further contain substances that increase the viscosity of the suspension including, for example, sorbitol, dextran and/or sodium carboxymethylcellulose. The suspension may also contain stabilizers. Carriers such as starches, sugars, microcrystalline cellulose, diluents, granulating agents, lubricants, binders, disintegrating agents, and the like may be used to form oral solid preparations such as powders or granules, microparticulates, nanoparticulates, capsules, gel capsules, sachets, tablets or minitablets. Each dosage form may include, apart from the essential components of the composition, conventional pharmaceutical excipients, diluents, sweeteners, flavoring agents, coloring agents and any other inert ingredients regularly included in dosage forms intended for oral administration (see, e.g., Remington's Pharmaceutical Sciences, 17th Ed., 1985). Because of their ease of administration, tablets and capsules are the preferred oral dosage units whereby solid pharmaceutical carriers are employed. Optionally, tablets may be coated by standard aqueous or nonaqueous techniques.

In a particularly preferred embodiment of the present invention, a pharmaceutical composition for oral administration to a mammalian subject is provided, comprising: a) at least one pheophorbide derivative compound as active ingredient; and b) a vehicle comprising a carrier such as TWEEN-80 (no less than 1%), and an appropriate bio-compatible solvent such as sterile saline or phosphate buffered saline etc.

Other carriers contemplated for use in the invention include, for example, Vitamin E TPGS (d-α-tocopheryl polyethylene glycol 1000 succinate, Eastman Chemical Co., Kingsport Tenn.); saturated polyglycolyzed glycerides such as GELUCIRE™ and LABRASOL™ products (Gattefossé Corp., Westwood, N.J.) which include glycerides of C₈-C₁₈ fatty acids; CREMOPHOR™ EL or RH40 modified castor oils (BASF, Mt. Olive, N.J.); MYRJ™ polyoxyethylated stearate esters (ICI Americas, Charlotte, N.C.); TWEEN™ (ICI Americas) and CRILLET™ (Croda Inc., Parsippany, N.J.) polyoxyethylated sorbitan esters; BRIJ™ polyoxyethylated fatty ethers (ICI Americas); CROVOL™ modified (polyethylene glycol) almond and corn oil glycerides (Croda Inc.); EMSORB™ sorbitan diisostearate esters (Henkel Corp., Ambler, Pa.); SOLUTOL™ polyoxyethylated hydroxystearates (BASF); and β-cyclodextrin.

It will be noted that several of the materials identified as carriers have also been found to be effective co-solubilizers, either alone or in combination with other viscosity-reducing agents, for certain other carriers. In general, any solvent in which pheophorbide derivative compounds are at least moderately soluble at body temperature or with gentle heating can be used as a co-solubilizer in the vehicle of the novel compositions.

Viscosity-reducing co-solubilizers contemplated for use include, e.g., PHARMASOLVE™ (N-methyl-2-pyrrolidone, International Specialty Products, Wayne, N.J.); MIGLYOL™ glycerol or propylene glycol esters of caprylic and capric acids (Hütls AG, Marl, Germany); polyoxyethylated hydroxystearates (e.g., SOLUTOL™ HS 15); TWEEN™ polyoxyethylated sorbitan esters; SOFTIGEN™ polyethylene glycol esters of caprylic and capric acids (Hüls AG); modified castor oils (such as CREMOPHOR™ EL or RH 40); vegetable oils such as olive oil, sesame oil, polyoxyethylated fatty ethers or modified castor oils; certain saturated polyglycolyzed glycerides (such as a LABRASOL™) citrate esters such as tributyl citrate, triethyl citrate and acetyl triethyl citrate; propylene glycol, alone or in combination with PHARMASOLVE™; ethanol; water; and lower molecular weight polyethylene glycols such as PEG 200 and 400.

The concentration of the active pheophorbide derivative compound in the composition may vary based on the solubility of the active agent in the carrier(s) or carrier(s)/co-solubilizer(s) system and on the desired total dose of pheophorbide derivative compound to be administered orally to the patient. The concentration of pheophorbide derivative compound may range from about 2 to about 500 mg/ml or mg/g of vehicle, and preferably from about 2 to about 50 mg/ml or mg/g.

Other suitable carriers may include mixtures of physiological saline with detergents, e.g., TRITON X-100® with solvents, such as dimethylsulfoxide (DMSO), or within liposomes. In all cases, any substance used in formulating a pharmaceutical preparation of the invention should be virus-free, pharmaceutically pure and substantially non-toxic in the amount used. One or more penetration enhancers surfactants and chelators may be included. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusid-ate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. Further penetration enhancers include polyoxyethylene-9-lauryl ether, polyoxyethylene-20-cetyl ether.

In another embodiment describing oral administration of pheophorbide derivative compounds, tablets containing the active agent is combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the pheophorbide derivative compounds may be combined with various sweetening or flavoring agents, coloring matter or dyes, and, if so desired, emulsifying and/or suspending agents as well, together with such diluents as water, ethanol, propylene glycol, glycerin and various like combinations thereof.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual compositions of the present invention, and the delivery means, and can generally be estimated based on EC₅₀'s found to be effective in in vitro and in vivo animal models.

Compositions and formulations for parenteral, intrathecal or intraventricular administration may include sterile aqueous solutions that may also contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation. The compositions of the present invention may contain additional pharmaceutically acceptable auxiliary substances as required to approximate physiological conditions and as necessary to prepare compositions for convenient administration, such as pH adjusting and buffering agents, and delivery vehicles. For example, tonicity adjustors may be added as needed or convenient. They include, but are not limited to, salts, particularly sodium chloride, potassium chloride, mannitol and glycerin, or any other suitable tonicity adjustor. Various buffers and means for adjusting pH may be used so long as the resulting preparation is pharmaceutically acceptable. Such buffers include acetate buffers, citrate buffers, phosphate buffers and borate buffers. Actual methods for preparing pharmaceutically administrable compounds will be known or apparent to those skilled in the art and are described in detail in, for example, Remington's Pharmaceutical Science, Mack Publishing Co., Easton, Pa. (1985), which is incorporated herein by reference.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like. Additional other cytotoxic, chemotherapeutic or anti-cancer agents contemplated for use include alkylating agents or agents with an alkylating action, such as cyclophosphamide (CTX; e.g. cytoxan®); anti-metabolites, such as methotrexate (MTX) and 5-fluorouracil (5-FU); antibiotics; other antitumor agents, such as paclitaxel and pactitaxel derivatives, the cytostatic agents, glucocorticoids and corticosteroids such as prednisone, leucovorin, folinic acid and other folic acid derivatives, and similar, diverse antitumor agents.

The present invention relates to providing an efficient and convenient method for obtaining a compound having therapeutic activity from a plant or plant part. The method comprises the steps of: obtaining the raw material, e.g., seeds, from the plant; extracting or exuding compounds from the raw material; identifying active compounds using in vitro assays, photodiode array detectors and mass spectrometry; separating active compounds using flash column chromatography; identifying active compounds using in vivo assays; fractionating active compounds using HPLC; screening active compounds using in vitro assays; purifying active compounds using high resolution chromatography; and identifying active compounds via a final in vivo assay.

The present invention relates to a method of preventing, treating tumors or tumor metastases in a patient, comprising: administering to said patient a therapeutically effective amount of a pharmaceutical composition comprising at least one pheophorbide derivative compound in pharmaceutically acceptable excipient, carrier or vehicle. In some embodiments, the present invention provides a method for reducing cellular proliferation comprising the step of exposing a pheophorbide derivative compound to cells. In some embodiments, the cellular proliferation is associated with cancer. In some embodiments, the cells are located in vivo in a subject (e.g., a human). In some embodiments, the cancer is pancreatic cancer, breast cancer, colon cancer, lung cancer, skin cancer or prostate cancer.

The present invention relates to a method of preventing and/or treating non-cancer diseases or conditions that result from changes in cellular proliferation or angiogenesis process. These non-cancer conditions may include but are not limited to benign hypertrophy of tissues, arthritis, retinal ailments, skin abnormalities, scar formation, cardiovascular diseases, gastrointestinal dysfunction, hematologic illness, immunological imbalance, allergies, gynecological and urological problems, bacterial infections etc. Diseases involving the angiogenesis process include ailments/conditions that result from too high or too low levels of blood vessel formation.

The following examples are provided to describe the invention in further detail.

### EXAMPLE 1

This Example describes preliminary studies wherein a traditional (cultural) preparation of the extract from the seeds of the plant Livistona chinensis was tested for anti-angiogenic and anti-tumor activities in in vitro and in vivo assays. The initial preparation was prepared by boiling 300 grams of Livistona chinensis seeds for approximately eight hours to a final volume of 300 ml of water.

The following assays were used for the evaluation of the extract material and isolated compounds. For in vitro experiments, the crude extract was filtered with a 0.2 μm cut-off. In vitro sample preparation of purified extracts for dispersion into cell culture included the use of 5-8% DMSO in phosphate buffer. For in vivo experiments, the crude extract was diluted with drinking water at a 1:4 ratio. For the preparation of purified extracts to be administered to mice, TWEEN-80 was used as a detergent due to its neutral taste, odor, and high safety approval. Samples were typically dissolved in 1-5% TWEEN-80 in distilled water. For subcutaneous samples, the Livistona extract was dissolved in sterile normal saline solution with 4% TWEEN-80.

In vitro proliferation assays. Human breast cancer cell lines were obtained from ATCC (American Tissue Type Culture Collection) and grown with RPMI and 10% FCS. These included ER (estrogen receptor)-negative MDA-MB-231 and ER-positive MCF-7 lines. Cells were plated onto 48-well culture plates at 5,000 cells/well and incubated at 37° C. in 5% CO₂ in RPMI and 10% FCS. On day three, one microCurie of [methyl-³H]thymidine (Amersham) was added to each well. Approximately 15 hours later, the cells were fixed with trichloroacetic acid, washed with ethyl alcohol and lysed with sodium hydroxide. After adding glacial acetic acid, the cell lysates' radioactivity was counted in scintillation solution, as an index of DNA synthesis.

Angiogenenesis assays. Primary endothelial cells were plated on tissue culture flasks coated with 1.5% gelatin and maintained in endothelial growth media (EGM: endothelial cell growth medium supplemented with 10 ng/ml hEGF (human epithelial growth factor) and 2% fetal calf serum (FCS). In in vitro proliferative assays, endothelial cells were plated onto gelatinized 48-well culture plates at 10,000 cells/well and incubated at 37° C. in 5% CO₂ in EGM. On day three, one microCurie of [methyl-³H]thymidine was added to each well. Approximately 15 hours later, the cells were fixed with trichloroacetic acid, washed with ethyl alcohol and lysed with sodium hydroxide. After adding glacial acetic acid, the cell lysates' radioactivity was counted in scintillation solution. Human umbilical vein endothelial cells (HUVEC) purchased from Clonetics (San Diego, Calif.) were used.

In vivo mouse studies. For the xenograft models, SCID mice were bred in a pathogen-free colony. 8-10 week old female mice were usually used for our studies. All mice were housed four per cage, and were fed ad libitum with sterilized food pellets and sterile water. The cell line tested was MDA-MB-231, which is ER-negative and considered among the most aggressive and metastatic laboratory models of human breast cancer. We injected 107 cancer cells subcutaneously, and administered treatments on the same day as the tumor injection. Control mice received vehicle, and experimental mice were given intraperitoneal injections or oral administration of drug(s). The mice were observed for any sign of toxicity, which may include change in behavior, appearance, hair, weight, skin or eye infections, shortness of breath, deficient movement, and premature death. At the end of the experiment at day 45 (or when the tumor is >1.5 cm, whichever comes first), the mice were sacrificed by CO₂. The endpoint was the primary tumor size, which was measured in three dimensions with calipers. The Student's t-test and ANOVA test were used for these experiments.

### Results

In vitro. We initially tested the crude extract obtained from the whole seed. Subsequently, we separated the inner “meaty” kernel and compared its extract with that obtained from the whole seed (note that the initial starting materials were the same 300 g of whole seeds for each extract type). Subsequent to weighing out 300 g of seeds, the inner kernels were removed and the hard shells discarded to derive the “kernel” extract).

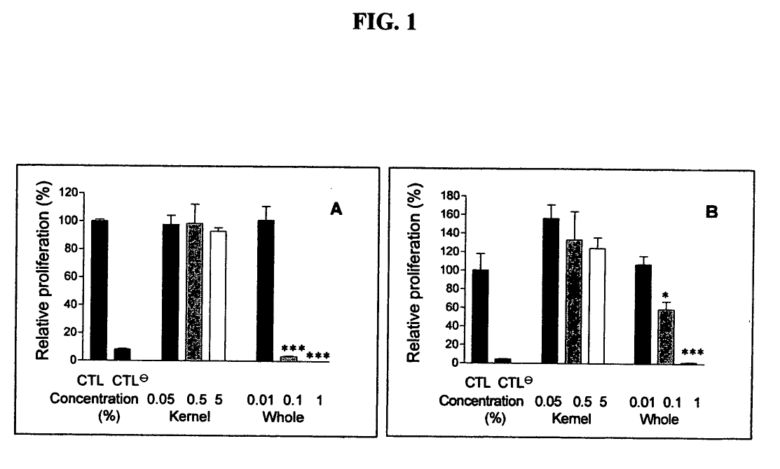

FIGS. 1A and 1B show that the major inhibitory effects of Livistona did not derive from the kernel portions. This is observed on the proliferation of two commonly used human breast cancer cell lines: highly aggressive ER-negative MDA-MB-231 (FIG. 1A), and less aggressive ER-positive MCF-7 (FIG. 1B).

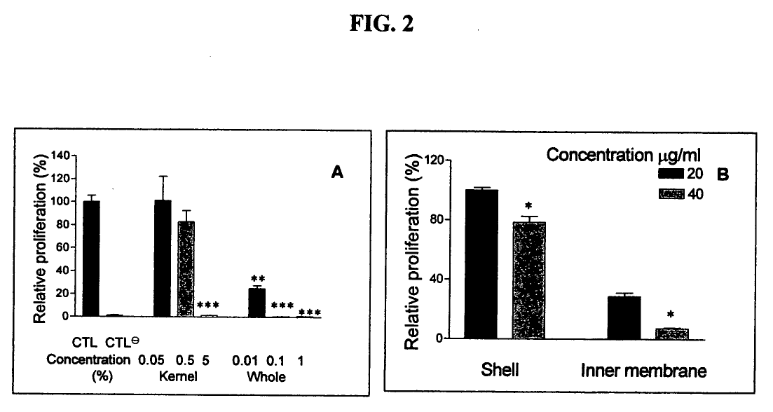

We repeated the same experiment with human umbilical vein endothelial cells (HUVECs, FIG. 2A), and observed the same phenomenon. HUVECs are often used for in vitro assessment of angiogenesis. We then separated the seeds into three components: 1) inner “meaty” kernel, 2) outer hard shell which includes a thin membrane attached to the inside of the hard shell, and 3) the thin membrane alone. FIG. 2B shows that the highest suppressive activity resides in the inner membrane of the shell. The results were expressed as the percentage of proliferation±standard error, in comparison with positive controls. Other experiments using breast cancer cells lines have confirmed the shell inner membrane as the location of maximal inhibitory activity (data not shown).

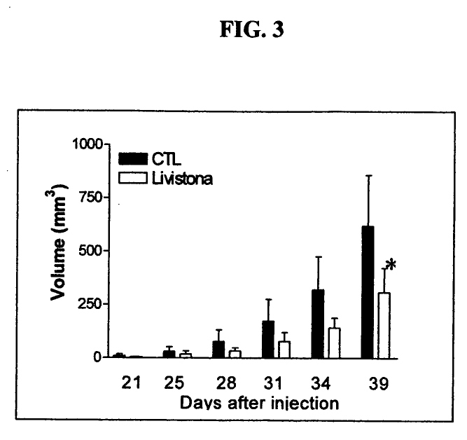

In vivo. In our mouse model of human breast cancer using the highly aggressive estrogen-independent ER-negative MDA-MB-231 xenografts, we observed smaller tumors in the mice that drank the Livistona crude extract obtained from the whole seeds (FIG. 3). The tumor size was measured with calipers twice weekly in three dimensions, and expressed as volume. We used analysis of variance (ANOVA) followed by the Bonferroni test to compare mean values of treated and control groups. There were no observed toxicities in the animals.

Based on this data, it was determined that the traditional preparation, using the complete seed shell, exhibited strong pharmacologic activity in proliferation assays and animal models, thus warranting further investigation into the efficacy of this extract, as well as the identification of any individual or combination of new chemical entities (NCE's) that may be responsible for such pharmacologic activity.

### EXAMPLE 2

In this Example, an isolation procedure designed to identify NCE's that may be responsible for the strong pharmacologic activity in proliferation assays and animal models within the Livistona extracts is provided.

In order to identify NCE's from complex mixtures, an interface of bio-directed fractionation and liquid chromatography coupled to photodiode array and mass spectrometry (LC-PDA-MS) and NMR was used to track the compounds at each stage of purification, and identify the active compounds. The employment of these analytical techniques allows the unique correlation of biological activity with mass, resonance and optical spectra as a means of fingerprinting active constituents along the purification route. This approach allows early signatures, such as the enrichment of m/z values, resonance, and uv/vis signals corresponding to the active constituents with the result that multiple activities can be tracked. Considering that many natural products may have more than one active chemical entity, this approach is ideal for identifying synergy amongst NCE's. Another important aspect of this approach is a periodic check for pharmacologic activity along the purification route. This assures that multiple constituents that may be active in vitro but not in vivo (a common occurrence) are distinguished and this is accomplished with small-scale animal xenograft studies. Once identification of compounds, or combination of compounds, responsible for the biological activity is completed, the process is scaled in order to test the compounds/components in in vitro tumorigenicity and angiogenesis assays. The best identified component(s) are then tested in tumor xenograft models.

The overall procedure for the isolation and identification thus consists of a series of chemical techniques along with extraction, fractionation and chromatographic separation. Each step in the schema is monitored by MS, NMR, and uv/vis (labeled as “analytical correlations”) with the benefit that the time for identification is reduced and pharmacologic activity due to synergy caused by multiple NCE's rather than just one NCE can be distinguished. Each of the isolation steps is based on bioassays of in vitro activities of the human breast cancer MDA-MB-231 cells and the human umbilical vein endothelial cells (HUVECs), and subsequent animal xenograft studies.

The overall isolation procedure is set forth in Scheme I below.

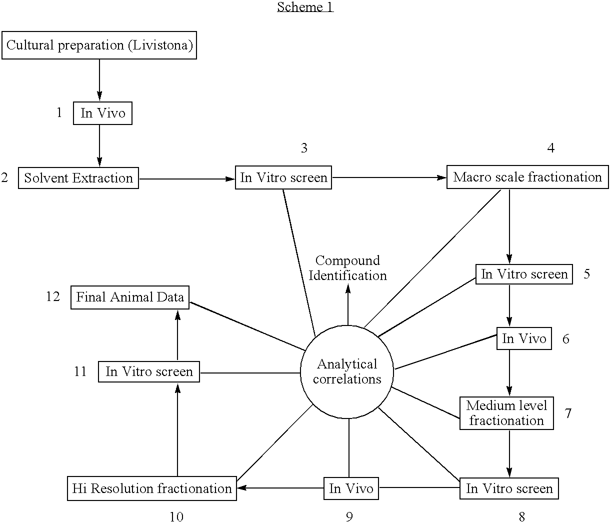

Step 1. Step 1 relates to the prior demonstration that a traditional preparation of the extract from the seeds of the plant Livistona chinensis exhibited strong pharmacologic activity in proliferation assays and animal models (Example 1).

Step 2. Extraction procedures using neat solvents are designed in order to simplify the complexity of the mixture.

Step 3. The neat solvent extracts are tested in vitro (using assays described in Example 1) to distinguish extracts possessing the greatest activity.

Step 4. Crude or large scale separation is performed by employing flash column chromatography.

Step 5. The separated fractions are profiled for biological activity using in vitro assays described above.

Step 6. The pharmacologic relevance of this activity is then screened for by using small scale in vivo xenograft models as described in Example 1.

Step 7. The pharmacologically active extract is subject to medium level fractionation using reverse phase preparative HPLC.

Steps 8-9. Further definition of the active constituents of the separation is then screened using the in vitro assays (step 8) and its pharmacologic relevance is again checked using the in vivo assays (step 9).

Steps 10-12. Final steps include: high-resolution chromatography (step 10); NCE identification via a final in vitro screen (step 11); and analytical characterization and final pharmacologic validation in animals for each compound (step 12).

### EXAMPLE 3

In this Example, compounds and compositions comprising the active constituents were isolated, and compounds possessing the principle Formula I were tested for their antineoplastic effect in vitro and in vivo using mice transplanted with cancer cells. The formulations were prepared in accordance with methods and procedures known and understood by those skilled in the art.

Following the protocol of Scheme 1, the following data was obtained.

Steps 2-3. Subsequent to determining that the traditional preparation, using the complete seed shell, had exhibited strong pharmacologic activity in proliferation assays and animal models, attempts were made to extract the activity into individual organic neat solvents. The use of a neat solvent is necessary because it retains the greatest activity and significantly reduces the complexity of the mixture vs. traditional aqueous/alcohol extraction procedures. The rationale for the use of neat solvents is that each solvent varies in its polarity index, and both polar protic as well as polar a-protic solvents can be employed so that compounds which are best soluble in a particular solvent go into that solution. The solvents were chosen to span a range of polarity indices ranging from 0.00 for hexanes to 9 for water. In this step, Livistona chinensis seeds were shelled, and the complete seed shells ground to a fine powder. The powder was extracted by stirring overnight in the particular solvent and subsequently filtered with Watman 5 paper and dried in vacuo. Weights of the residue were determined and tested in vitro using both MB-MDA-231 breast cancer cell line and HUVECs as described above. Our findings indicated that acetonitrile with 1% water gave the most potent activity and thus this solvent (the highest polarity index for the class of polar a-protic solvents) was used to extract the seed shells henceforth.

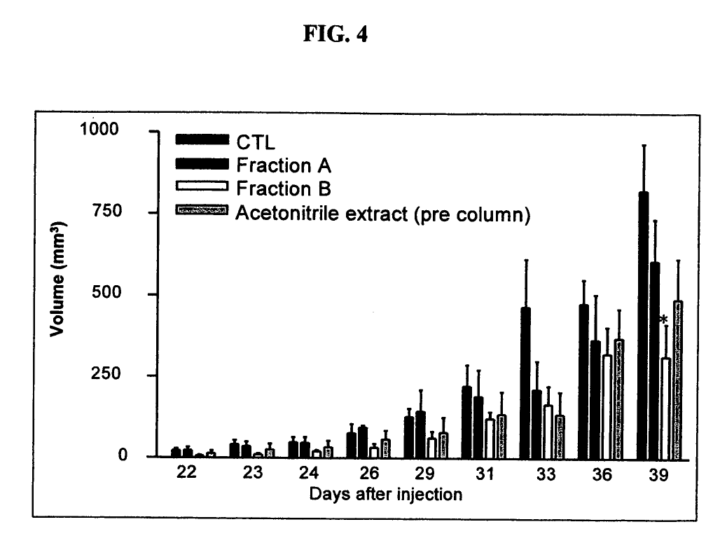

Steps 4-6. Crude separation of the extract using normal phase column chromatography: Using silica gel flash column chromatography and a mobile phase of CHCl₃/MeOH 15/1 v/v, separation of the acetonitrile extract was possible and no material was retained on the column. Analysis of the eluent by thin layer chromatography (TLC) yielded two major groups of compounds eluting early and late in the chromatogram, which correlated with in vitro biological activity. Fractions were dried down in vacuo. Fractions were prepared for in vitro assays by dissolving them in phosphate buffer with 5% DMSO and the assays were performed as described above. As stated previously, the in vitro studies distinguished two main regions, eluting early and late in the chromatogram, that possessed significant inhibitory activity toward cancer cell proliferation in vitro. The later fractions, however, showed greater activity than the former. Henceforth, the latter fractions will be referred to as “fraction B” and the former as “fraction A.” In order to screen the extract to correlate in vitro activity with pharmacologic activity, the aforementioned chromatographic separation was pooled into two groups corresponding to the two areas of activity. Three groups of mice were chosen to receive oral preparations by gavage of fraction A, B, total acetonitrile extract (pre column), or vehicle (control). After injection with 107 MDA-MB-231 cells subcutaneously in the flank, groups of four mice each received 40 mg/kg of fraction A, 10 mg/kg of fraction B, 40 mg/kg of the acetonitrile extract, and vehicle alone (distilled water and 5% TWEEN-80). Fraction B was administered at ¼^{th} the concentration of both fraction A and the total extract (acetonitrile) due to limited supply. The results are represented in FIG. 4. Fraction B demonstrated the greatest activity (38% control) using ¼^{th} the amount of the other two study groups. This data indicates that the pharmacologic activity observed from the traditional preparation is contained within the acetonitrile extract. Furthermore, the data indicates that the pharmacologic effect is eluted off the column during this crude separatory step. This step effectively reduces the weight of material approximately 1,000 fold and fraction B demonstrates greater activity than fraction A or the pre-column extract, even at 25% the concentration of the other samples.

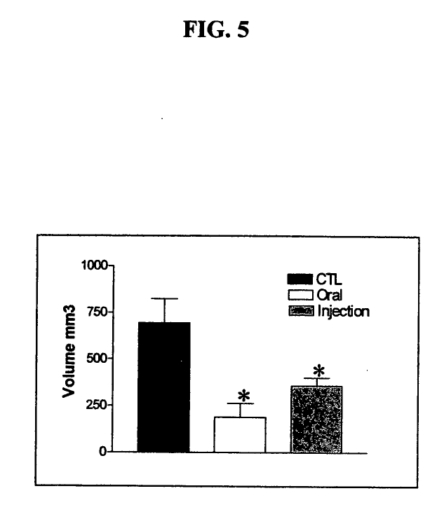

Steps 7-9. Following the crude fractionation procedure, a medium level separation (preparative RP HPLC—for this HPLC step, two columns was employed: a RP 250×21.4μ and 4μ 250×10 mm column that is capable of taking 100% aqueous solvents and a normal phase column if needed. Solvent system included the use of the volatile buffer NH₄OAc pH 7.5 to prevent interference with mass spectrometry and biological analysis) along with electrospray mass spectrometry coupled to photo diode array and bio-directed fractionation was performed to further define the biological activity. Correlating in vitro activity with mass and PDA (photo diode array) signals yielded a particular series of signals, which had potent in vitro activity. In order to again verify if the activity associated with these signals was correlated with the pharmacologic activity witnessed by the total extract, another small scale in vivo study was performed using two routes of entry: oral administration by gavage and subcutaneous injection. Oral administration was by gavage at 10 mg/kg five days per week, and subcutaneous injection was administered at 3 mg/kg three days per week. The results are summarized in FIG. 5.

This data shows that the pharmacologically active constituent(s) is/are retained in this step. The approximate reduction in tumor volume is 72% in the oral administration group. The subcutaneous group showed approximately 55% tumor volume reduction. The significance of this result is that the active constituent(s) do not require activation in the gut or first pass through the liver as a necessary component of their pharmacologic activity. This is important when we consider the applicability to human studies. In all animal studies, toxicity has not been observed up to 10 fold the dose shown in FIG. 5 (data not shown).

### EXAMPLE 4

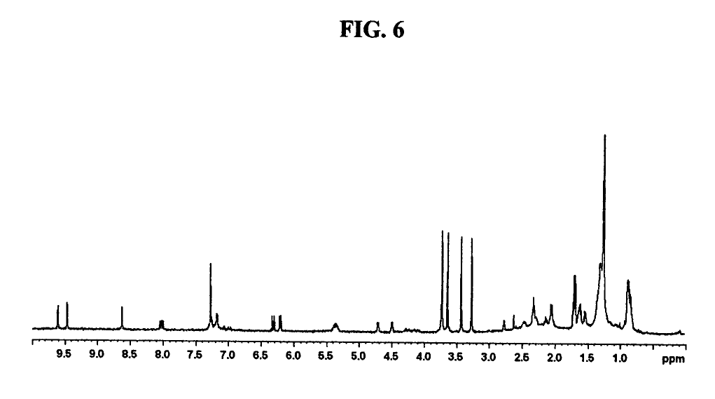

Step 10-12. Identification of active component(s): ¹H nuclear magnetic resonance (NMR), ultraviolet (UL/Visible) spectroscopy and high-resolution mass spectrometry (HR-MS) were performed on each compound isolated. These spectroscopic methods are used for structural elucidation. Proton NMR and the use of a proprietary compound database revealed multiple compounds of general Formula I. The ¹H proton spectra for a specific pheophorbide derivative compound (in neat deuterated DMSO) is shown in FIG. 6. This compound is referred to herein as AM-101. FIG. 6 consists of spectra of a pheophorbide derivative compound having the principle Formula I wherein R₁═R₄═CH₃; R₂═CH₂COOCH₃; R₃═COOH; X₁═O; and X₂═OH.

Low resolution mass spectrometry was employed using electrospray ionization mass spectrometry (ESI-MS) on a Perkin-Elmer Sciex (Thornhill, Canada) API III triple quadrupole mass spectrometer tuned and calibrated in the positive ion mode as described (Glasgow, B. J., Biochemistry, vol. 37, p. 2215-25, 1998).

Fourier-transform mass spectrometry (FT-MS) was employed to obtain high resolution mass spectra for AM-101. The M+Na⁺ salt had a calculated mass of: 631.2533 Da and the measured mass was observed to be 631.2527±0.00159 Da with the elemental composition being C₃₅H₃₆N₄NaO₆⁺. Thin layer chromatography using CHCl₃/Methanol 15/1 gave a single spot with an R_{f} of 0.42.

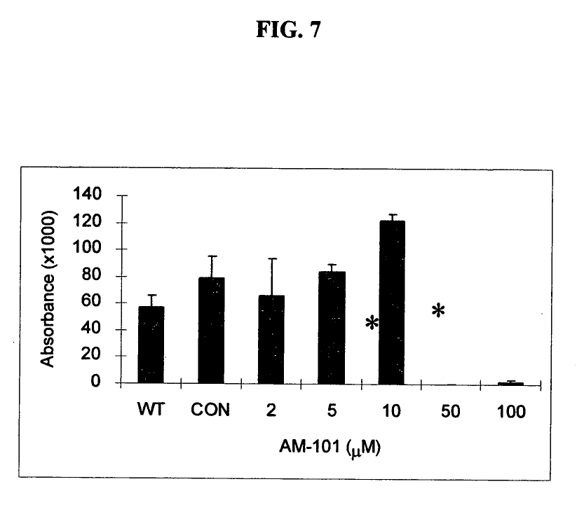

The AM-101 compound was further tested on the human breast cancer cell line MDA-MB-231 for its inhibitory activity. The CellTiter 96-Aqueous non-radioactive cell proliferation assay from Promega (Madison, Wis.) was used according to the manufacturers' instructions. As depicted in FIG. 7, the AM-101 compound of the present invention powerfully inhibits the proliferation of human breast cancer cells in the absence of light. It was thus determined that AM-101 is the active component(s) from the plant Livistona chinensis and is a light independent, highly active anti-tumor agent.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. Pheophorbide derivative compounds having the general Formula I: 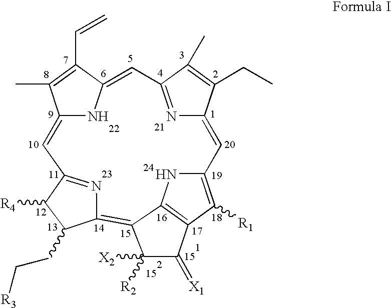
wherein
R₁═R₄═CH₃; R₂═CH₂OH, COOH, CHO, COOCH₃, alkyl or ester or ether linked alkyl group containing alkyl substitutents such as (CH₂)ₙCH₃ where n is between 0 and 24 and may contain 0, 1, 2 or 3 double bonds and 0 or more hydroxy moieties, and may contain one or more of the following esters selected from hemisuccinate, choline, phosphate, amino acid, phosphoryloxymethylcarbonyls, dimethylaminoacetate, phosphonate, and N-alkoxycarbonyl and phosphoryloxymethyloxycarbonyl derivatives; R₃═OH, COOH, COOCH₃, alkyl group or ester linked alkyl group containing alkyl substitutents such as (CH₂)ₙCH₃ where n is between 0 and 24 and may or may not contain 0, 1, 2 or 3 double bonds and 0 or multiple hydroxy moieties, and may contain one or more of the following esters selected from hemisuccinate, choline, phosphate, phosphoryloxymethylcarbonyls, amino acid, phosphonate, dimethylaminoacetate, and N-alkoxycarbonyl and phosphoryloxymethyloxycarbonyl derivatives; X₁═O, S, or the reduced form at C15¹ yielding OH, SH, and any ester or ether derivatives thereof; X₂═H, OH, OCH₃, OCH₂CH₃, OAc, SH, Cl, and F; and wherein said compounds are photo-independent cytotoxic agents able to inhibit tumor growth in a mammal.

2. A pharmaceutical composition comprising a pheophorbide derivative compound of claim 1, in a pharmaceutically acceptable excipient, carrier or vehicle.

3. A pharmaceutical composition of claim 2, additionally comprising one or more other anti-cancer agents.

4. A pharmaceutical composition of claim 3, wherein said other anti-cancer agent is selected from alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

5. A pharmaceutical composition for oral administration to a mammalian subject, comprising:
a) a pheophorbide derivative as active ingredient; and
b) a vehicle comprising:
i) TWEEN-80 at least 0.01% or as much as 10% by volume in a biologically compatible solvent selected from sterile water, PBS and normal saline; and
ii) carriers comprising at least 1-30% Vitamin E TPGS with various mixtures selected from mixtures of ethanol polyethylene glycol, and propylene glycol.

6. A method for obtaining a compound having therapeutic activity from a plant or plant part, comprising the steps of:
a) obtaining the raw material from the plant;
b) extracting or exuding compounds from the raw material;
c) identifying active compounds using in vitro assays;
d) separating active compounds using flash column chromatography;
e) identifying active compounds using in vivo assays;
f) fractionating active compounds using HPLC;
g) screening active compounds using in vitro assays;
h) purifying active compounds using high resolution chromatography; and
i) identifying active compounds via a final in vivo assay.

7. A method of preparing a pharmaceutical composition having therapeutic activity, comprising the steps of:
a) obtaining a purified composition comprising the active compound from a plant;
b) adding a detergent or carrier agent in a neat solvent of which said active compound is readily dissolved into to form a mixture;
c) drying down said mixture to form a dried mixture;
d) resuspending said dried mixture in a biologically compatible solvent using sonication and rigorous mixing to form a fully resuspended composition.

8. A method of preparing a pharmaceutical composition having therapeutic activity, comprising the steps of:
a) obtaining a purified composition comprising the active compound from synthetic or semi-synthetic means;
b) adding a detergent or carrier agent in a neat solvent of which said active compound is readily dissolved into to form a mixture;
c) drying down said mixture to form a dried mixture;
d) resuspending said dried mixture in a biologically compatible solvent using sonication and rigorous mixing to form a fully resuspended composition.

9. A method for treating tumors or tumor metastases in a patient, comprising: administering to said patient a therapeutically effective amount of a pharmaceutical composition comprising at least one pheophorbide derivative compound in pharmaceutically acceptable excipient, carrier or vehicle.

10. A method of claim 9, wherein the patient is a human that is being treated for cancer, in preventive and/or active disease situations.

11. A method of claim 9, wherein the tumors or tumor metastases to be treated are selected from lung cancer, colorectal cancer, NSCLC, bronchioloalviolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors and tumor metastases

12. A method of claim 9, wherein the tumors or tumor metastases are refractory.

13. A method of claim 9, wherein the tumors or tumor metastases to be treated are NSCLC tumors or tumor metastases.

14. A method of claim 9, additionally comprising administering one or more other anti-cancer agents.

15. A method of claim 9, wherein said composition is administered to said patient by oral administration.

16. A method of claim 9, wherein said composition is administered to said patient by a route selected from intravenous, subcutaneous, topical, and transdermal.

17. A method of claim 9, wherein said composition is administered to prevent and/or treat non-cancer diseases or conditions that result from changes in cellular proliferation selected from benign hypertrophy of tissues, arthritis, retinal ailments, skin abnormalities, scar formation, cardiovascular diseases, gastrointestinal dysfunction, hematologic illness, immunological imbalance, allergies, gynecological and urological problems.

18. A method of claim 9, wherein said composition is administered to prevent and/or treat non-cancer diseases or conditions that result from changes in angiogenesis process selected from ailments/conditions that result from too high or too low levels of blood vessel formation.

19. A method of claim 9, wherein said composition is administered to treat one or more infections caused by one or multiple agents selected from bacteria, fungi, viruses, mycobacteria, and yeast.
